(19) United States
(12) Patent Application Publication
(10) Pub. No.: US 2015/0374851 A1
(43) Pub. Date: Dec. 31, 2015

(54) AAV VECTORS EXPRESSING SEC10 FOR TREATING KIDNEY DAMAGE

(71) Applicant: The Trustees of the University of Pennsylvania, Philadelphia, PA (US)

(72) Inventors: Joshua H. Lipschutz, Bala Cynwyd, PA (US); Jean Bennett, Bryn Mawr, PA (US); Daniel C. Chung, West Chester, PA (US)

(21) Appl. No.: 14/789,159

(22) Filed: Jul. 1, 2015

Related U.S. Application Data
(63) Continuation of application No. 13/499,212, filed on Jun. 7, 2012, now abandoned.
(60) Provisional application No. 61/247,746, filed on Oct. 1, 2009.

(51) Int. Cl. A61K 48/00; C12N 7/00

(52) U.S. Cl. CPC A61K 48/0075; C12N 7/00; C12N 2710/10032

(57) ABSTRACT

A method for enhancing repair of damaged mammalian tubular epithelial cells involves delivering to the tubular epithelial cells of a subject in need thereof a composition comprising an adeno-associated virus (AAV) comprising an AAV capsid having an amino acid sequence of a selected AAV serotype, and a minigene having AAV inverted terminal repeats and a Sec10 gene operatively linked to regulatory sequences that direct expression of Sec10 in the epithelial cells. In one embodiment, delivery is accomplished by retrograde intrauretal injection. In an embodiment the AAV vector includes a capsid of AAV serotype 2/8. Therapeutic compositions containing such AAV are provided.

### CROSS-REFERENCE TO RELATED APPLICATIONS

This application is a continuation of U.S. patent application Ser. No. 13/499,212, filed Mar. 29, 2012 (371 documents completed Jun. 7, 2012) which is a national stage of International Patent Application No. PCT/US2010/050852, filed Sep. 30, 2010, which claims the benefit of the priority of U.S. Provisional Patent Application No. 61/247,746, filed Oct. 1, 2009 (expired), which applications are incorporated herein by reference.

### BACKGROUND OF THE INVENTION

Acute Tubular Necrosis (ATN), a form of acute kidney injury (AKI) is characterized by the death of the tubular epithelial cells of the kidney. AKI/ATN affects approximately 500,000 patients each year. AKI/ATN is a leading cause of acute kidney failure which is present in 5% of all patients admitted to the hospital. AKI/AKN can have many causes including trauma, ischemia/reperfusion injury of the kidney due to clinical testing or vascular or other surgeries, exposure to toxins, such as the iodinated contrast agents used for CT studies, and other clinical tests, stress, hypertension, and surgery. For example, ischemia/reperfusion results in apoptotic and necrotic death of tubular epithelial cells, impairs renal function, and causes ATN. Because there are approximately 28 million MRI procedures performed annually in the US, AKI/AKN in hospitalized patients is a significant and increasing problem in the US.

In many cases of AKI/ATN, the damaged cells are able to repair themselves. Severely damaged kidneys sustaining ischemia/reperfusion injury typically, though not always, recover from this insult within days to weeks. Post-ischemic restoration of renal tubular epithelial cells occurs because cells surviving the injury divide, differentiate, and finally mature into functional epithelial cells. However, in severe cases, AKI/ATN can lead to acute renal failure. In renal failure, tubular damage is not repaired. Mortality rates in affected patients remain very high (>50%). Additionally, recent studies have demonstrated that despite recovery following ischemia/ reperfusion, the kidneys undergo mild permanent changes, such as expansion of the interstitial space, depending on the severity of the ischemic damage.

There are currently no approved therapies for AKI/ATN. Medical management of AKI/ATN has traditionally consisted of supportive care, with renal replacement therapy, i.e., transplantation, implemented for the most severe cases. There is, therefore, a need in the art for safe therapeutic and prophylactic compositions and methods to improve, accelerate, or potentially replace, the native recovery process of injured tubular epithelial cells affected by AKI/ATN.

### SUMMARY OF THE INVENTION

Described herein are compositions and methods to improve, accelerate, or potentially replace, the native recovery process of injured tubular epithelial cells affected by AKI/ATN.

In one aspect, a method for enhancing repair of damaged mammalian tubular epithelial cells is provided, which involves delivering to the damaged tubular epithelial cells a composition permitting overexpression of Sec10 to the cells. In one embodiment, such a composition comprises an adeno-associated virus (AAV) vector. In an embodiment, delivery is accomplished by retrograde ureteral injection.

In another aspect, a method for treating a mammalian subject in danger of developing damage to the subject's tubular epithelial cells is provided, which involves delivering to the tubular epithelial cells a composition permitting overexpression of Sec10 to the cells. In one embodiment, such a composition comprises an adeno-associated virus (AAV) vector. In an embodiment, delivery is accomplished by retrograde ureteral injection.

In another aspect, a method for enhancing repair or regeneration of mammalian renal tubular epithelial cells involves delivering to the kidney of a subject in need thereof via endoscopic retrograde ureteral injection a composition comprising an adeno-associated virus (AAV) comprising an AAV capsid having an amino acid sequence of a selected AAV serotype, and a minigene having AAV inverted terminal repeats and a Sec10 gene operatively linked to regulatory sequences that direct expression of Sec10 in the kidney's tubular epithelial cells. In one embodiment, the selected AAV serotype is AAV 8 or a chimeric AAV2/8.

In still another aspect, a composition for enhancing repair or regeneration of mammalian renal tubular epithelial cells is provided, which includes an adeno-associated virus (AAV) comprising an AAV capsid having an amino acid sequence of a AAV2/8 or AAV8 serotype, and a minigene having AAV inverted terminal repeats and a human Sec10 gene operatively linked to regulatory sequences that direct expression of Sec10 in a subject's epithelial cells, in a physiologically compatible carrier.

In still another aspect is a use of a composition permitting overexpression of SEC10 for, or in the preparation of a medicament for, enhancing repair or regeneration of mammalian renal tubular epithelial cells.

Other aspects and advantages of the invention are described further in the following detailed description of the preferred embodiments thereof.

### BRIEF DESCRIPTION OF THE DRAWINGS

FIG. 1A are four photomicrographs taken with Olympus microscope showing that Sec10-overexpression resulted in reduced loss of dome. Normal (wild-type) and hSec10-overexpressing (Sec10) MDCK cells were grown on plastic culture dish to the point of confluence and formation of dome. The confluent grown cells were treated with 0 or 1 mM H₂O₂ (which is an in vitro model of ischemia/reperfusion injury involving oxidative stress) for 30 minutes. Domes were observed on light microscope. Arrows indicate damaged domes.

FIG. 1B is a graph plotting the numbers of damaged and intact domes from FIG. 1A counted under microscope 30 minutes after treatment of 1 mM H₂O₂. Values represent % damaged dome (damaged dome/(intact+collapsed domes)×100). Values represent mean±SE. *, p<0.05 versus respective control. #, p<0.05 versus wild-type.

FIG. 2A is a bar graph showing that Sec10-overexpression resulted in increased transepithelial electric resistance (TER) after hydrogen peroxide treatment. Control (wild-type) and Sec10-overexpressing MDCK cells were grown on the Transwell filter over 7 days and then treated with either vehicle or 1 mM of H₂O₂. TER of normal (Wild-type) and Sec10-overexpressing (Sec10) type II MDCK cells was measured.

FIG. 2B is a bar graph showing that Sec10-overexpression inhibited reduction of TER after hydrogen peroxide treatment. Cells were treated with 1 mM H₂O₂ and then TER was measured at the indicated times. Values present the mean±SE (n=6-12). *, p<0.05 versus respective control. #, p<0.05 versus Sec10.

FIG. 3A-B are micrographs of gels showing that Sec10-overexpression resulted in increased phosphorylation of extracellular signal-regulated kinase (ERK).

FIG. 3A is a photomicrograph of a Western gel produced from phosphor-ERK expression in the MDCK cells (Control; wildtype) cultured on plastic dishes at confluence (5d) then treated with no H₂O₂ for 30 minutes, and harvested for Western blot analysis after lysis in SOS buffer. Equal amounts of protein were loaded in each lane as determined by bicinchoninic (BCA) assay, and Western blot was performed using antibodies against phosphorylated (active) ERK and total ERK. Phosphorylated ERK levels were higher in the Sec10 overexpressing cells compared to control cells, while total ERK levels were unchanged. The lanes are all from the same gel; however, the control and Sec10 OE lanes were separated on the gel.

FIG. 3B shows the gel produced from phosphor-ERK expression in the cells cultured on plastic dishes at confluence then treated with no or 0.5 or 1 mM of H₂O₂ for 30 min and harvested for Western blot analysis. Equal amounts of protein were loaded in each lane as determined by BCA assay, and Western blot was performed using antibodies against phosphorylated (active) ERK and total ERK.

FIG. 3C shows the gel produced from phosphor-ERK expression in the cells cultured on transwell filter culture dishes at confluence, then treated with 0 or 0.5 or 1 mM of H₂O₂ for 30 min and harvested for Western blot analysis. Equal amounts of protein were loaded in each lane as determined by BCA assay, and Western blot was performed using antibodies against phosphorylated (active) ERK and total ERK.

FIG. 4A is a photograph of a Western gel produced in an experiment to demonstrate that inhibition of ERK activation accelerated decrease of TER induced by hydrogen peroxide. Normal and hSec10-overexpressing (Sec10) MDCK cells were grown on transwell filter culture dish at confluence, incubated in either vehicle or 10 μM of U0126 (an inhibitor of ERK activation) for 30 min, and then treated with 1 mM of H₂O₂ for 30 min. After 30 min of incubation in U0126 cells were harvested and used to detect the levels of phosphorylated ERK. Cells were lysed in SDS buffer. Equal amounts of protein were loaded in each lane as determined by BCA assay, and Western blot was performed using antibodies against phosphorylated (active) ERK and total ERK.

FIG. 4B is a graph showing the results of the experiment of FIG. 4A for wild-type MCDK cells. TER was measured at the indicated time points. Values present the mean±SE (n=6). *, p<0.05 versus respective 0 min. #, p<0.05 versus Sec10.

FIG. 4C is a graph showing the results of the experiment of FIG. 4A for Sec10-overexpressing MCDK cells. TER was measured at the indicated time points. Values present the mean±SE (n=6). *, p<0.05 versus respective 0 min. #, p<0.05 versus Sec10.

FIG. 5A is a graph showing that ERK inhibition accelerated decrease of TER induced by hydrogen peroxide and inhibited recovery of TER. Normal and hSec10-overexpressing (Sec10) MDCK cells were grown on transwell filter culture dish at confluence. Cells were treated with U0126, an ERK inhibitor, 30 min before H₂O₂ treatment and then 1 mM H2O2. TER was measured at the indicated time points. Values present the mean±SE (n=6).

FIG. 5B is a graph similar to that of FIG. 5A. Normal and hSec10-overexpressing (Sec10) MDCK cells were grown on transwell filter culture dish at confluence. Cells were treated with H₂O₂ treatment for 30 min and incubated in 10 μM U0126. TER was measured at the indicated time points. Values present the mean±SE (n=6).

FIGS. 6A and 6C show two photographs of an intact and damaged cyst, respectively. hSec10-overexpression resulted in decreased damage of cysts induced by hydrogen peroxide treatment. Normal and hSec10-overexpressing (Sec10) MDCK cells were grown on collagen-matrix for 12-14 days as described herein and then treated with 1 mM of hydrogen peroxide for 30 min. Some cells were treated with 10 μM U0126 30 min before the treatment of hydrogen peroxide. After the treatment cells were fixed with 4% paraformaldehyde and then stained with F-actin phalloidin-conjugated cy3. Numbers of damaged cysts were counted using fluorescence microscope. Damaged cysts were evaluated by collapse of cysts and/or loss of cell polarity as seen in F-actin phalloidin staining.

FIG. 6B is a graph showing for FIGS. 6A and 6C the numbers of damaged and intact cysts counted under fluorescence microscope. Values indicates mean±SE (n=3). *, p<0.05 versus respective control. #, p<0.05 versus Sec10.

FIG. 7A are photomicrographs of gels showing that ischemia and reperfusion resulted in changes of plasma creatinine concentration in the mouse kidneys. Mice were subjected to 30 min of ischemia and reperfusion for indicated time periods. Blood was harvested to determine concentration of plasma creatinine (n=4-7 ), Sec8, PCNA and GAPDH expression by Western blot analysis. GAPDH was used for marker of equal loading. Values present mean±SE. *, p<0.05 versus baseline, 0 day.

FIG. 7B is a graph showing levels of Sec8 and proliferating cell nuclear antigen (PCNA). Mice were subjected to 30 min of ischemia and reperfusion for indicated time periods. Kidneys were harvested to determine levels of plasma creatinine (n=4-7) from the experiment of FIG. 7A. Values present mean±SE. *, p<0.05 versus baseline, 0 day.

FIG. 8A is a graph showing quantification that demonstrates the increased rate and efficiency of mature cyst formation in hSec10-overexpressing cell cysts, as described in Example 3 below.

FIG. 8B is a bar graph showing quantification of the number of tubules per cyst, as described in Example 3 below. hSec10=human Sec10. Clones 1, 2, and 3 refer to different stable hSec10-overexpressing cell lines. Bar=30 μm.

FIG. 9 is a bar graph showing the results of exocyst expression in mouse embryonic kidneys. RNA was harvested from mouse embryonic kidneys and reverse transcription (RT) was performed. Real-time PCR was performed using unique primers for the different exocyst proteins. Expression of exocyst complex member Exo70 was representative, and is shown here because of Exo70 was run concomitant with Wnt-4. The results were normalized to f3-tubulin.

FIG. 10A is a graph quantifying Exocyst expression in kidneys following ischemic injury. C57BU6 male mice were subjected to 30 minutes of ischemia by occlusion of the renal pedicles with a microaneurysm clamp. Blood urea nitrogen (BUN) levels were determined at the indicated time points following release of the clamps. The values presented are the means±the S.E. (n=4-7 per time point). *, P<0.05 when compared to the BUN at 0 day.

FIG. 10B are micrographs of gels showing expression of exocyst component Sec8, proliferating cell nuclear antigen (PCNA), and Na/K-A TPase in the kidneys of mice subjected to 30 min of ischemia. Expression is shown over 84 hours post ischemia/reperfusion (left) compared to expression over 16 days post ischemia/reperfusion (right). Sec8, PCNA, and Na/K-ATPase expression were determined by Western blot using anti-Sec8, -PCNA, and -Na/K-ATPase antibodies. Western blot with antibody against the housekeeping protein GAPDH was used as a loading control. As PCNA, a marker of tubular proliferation, decreased between days 8 and 16 following ischemia and reperfusion, and tubules began to re-differentiate as seen by the re-expression of the Na/K-ATPase transporter in the lower gels, the exocyst component Sec8 increased. Kidneys were harvested at the indicated times after reperfusion.

### DETAILED DESCRIPTION OF THE INVENTION

Therapeutic and prophylactic methods employing compositions for the delivery and over-expression of Sec10 in renal tubule epithelial cells are provided to enhance or improve the natural recovery process of tubular epithelial cells from damage due to injury or disorder. These methods can in one embodiment restore proper kidney function after such damage more quickly than current modalities and can limit or prevent further or future injury to the kidney due to disease or environmental causes.

A method for enhancing repair of damaged mammalian tubular epithelial cells is provided, which involves delivering to the damaged tubular epithelial cells a composition permitting overexpression of Sec10 to the cells. In one embodiment, a method for enhancing repair of damaged mammalian tubular epithelial cells involves delivering to the damaged renal tubular epithelial cells of a mammal, preferably a human, a composition comprising an adeno-associated virus (AAV) comprising an AAV capsid having an amino acid sequence of a selected AAV serotype, and comprising a Sec10 gene operatively linked to regulatory sequences that direct expression of Sec10 in subject's cells.

In another aspect, a method for enhancing repair or regeneration of mammalian renal tubular epithelial cells involves delivering to the kidney of a subject in need thereof via endoscopic retrograde ureteral injection a composition comprising an adeno-associated virus (AAV) comprising an AAV capsid having an amino acid sequence of a selected AAV serotype, and a minigene having AAV inverted terminal repeats and a Sec10 gene operatively linked to regulatory sequences that direct expression of Sec10 in the kidney's tubular epithelial cells. In one embodiment, the selected AAV serotype is AAV 8 or a chimeric AAV2/8.

In another embodiment, a method for enhancing repair or regeneration of mammalian renal tubular epithelial cells comprising delivering to the kidney of a subject in need thereof via endoscopic retrograde ureteral injection a composition comprising an adeno-associated virus (AAV) comprising an AAV capsid having an amino acid sequence of a selected AAV2/8 serotype, and a minigene having AAV inverted terminal repeats and a Sec10 gene operatively linked to regulatory sequences that direct expression of Sec10 in the kidney's tubular epithelial cells.

In another aspect, a method for treating a mammalian subject in danger of developing damage to the subject's tubular epithelial cells is provided, which involves delivering to the tubular epithelial cells a composition permitting overexpression of Sec10 to the cells. In another embodiment, a method for preventing tubular epithelia damage in those at risk for ATN or another kidney ailment or exposure to an environmental source of kidney damage involves delivering in proximity to renal tubular epithelial cells of a mammal, preferably a human, a composition comprising an adeno-associated virus (AAV) comprising an AAV capsid having an amino acid sequence of a selected AAV serotype, and a comprising a Sec10 gene operatively linked to regulatory sequences that direct expression of Sec10 in the subject's cells and overexpressing Sec10 at the site of the renal epithelial cells. The therapeutic compositions can be those used in the method for enhancing repair of damaged tubule epithelium. However in this embodiment, the composition is provided to a subject prior to occurrence or substantial occurrence of damage to the renal tubule epithelial cells.

In still another aspect, a therapeutic composition for such use is provided, which includes an adeno-associated virus (AAV) comprising an AAV capsid having an amino acid sequence of a AAV2/8 or AAV8 serotype, and a minigene having AAV inverted terminal repeats and a human Sec10 gene operatively linked to regulatory sequences that direct expression of Sec10 in the subject's epithelial cells, in a physiologically compatible carrier.

The various components of the methods and compositions for therapeutic or prophylactic treatment of subjects having, or in danger of having, ATI/ATN or other kidney diseases or exposures to environmental causes of renal tubule epithelial damage are discussed in detail and exemplified below.

### A. THE MAMMALIAN SUBJECT

As used herein, the term “mammalian subject” or “subject” includes any mammal in need of these methods of treatment or prophylaxis, including particularly humans. Other mammals in need of such treatment or prophylaxis include dogs, cats, or other domesticated animals, horses, livestock, laboratory animals, etc. In one embodiment, the mammalian subject has damaged tubule epithelial cells due to Acute Kidney Injury (AKI). In another embodiment, the mammalian subject has damaged tubule epithelial cells due to Acute Tubule Necrosis (ATN). In another embodiment, the subject has autosomal dominant polycystic disease. In still another embodiment, the subject is anticipating surgery or transplantation, or has had a kidney transplant. In still other embodiments, the subject in need of the method and therapeutic compositions described herein has any other kidney ailment that is characterized by damaged renal tubule epithelial cells. In a further embodiment, the subject is anticipating potential damage to the renal tubule epithelium, such as a subject scheduled for clinical diagnostic treatments normally damaging to the kidney, such as MRI or other therapeutic regimen employing dyes or toxic substances. In a further embodiment, the subject is anticipating potential damage to the renal tubule epithelium due to a genetic disorder providing a predisposition to kidney damage. Other subjects who would find use in the methods described herein are those anticipating exposure to possible kidney-damaging toxins, infectious diseases and the like. This method can also be used preemptively in those subjects at high risk for developing ATN or another kidney disease.

The methods and therapeutic compositions described herein involving overexpression of Sec10, may accelerate recovery from kidney damage or protect these subjects from developing kidney ailments.

### B. EXOCYST AND SEC10

The exocyst is a 750 kD complex comprised of eight subunits, i.e., Sec3, Sec5, Sec6, Sec8, Sec10, Sec15, Exo70, and Exo84 [Grindstaff K K, et.al., 1998 Cell 93: 731-740; Rogers K K, et.al., 2003 Kidney Int63: 1632-1644; and Terbush DR, et.al., 1996 EMBO J15: 6483-6494). The exocyst is a central component of the secretory pathway, which is involved in the synthesis and delivery of secreted and membrane proteins and in cell to cell contact. This pathway is absolutely essential for many cellular functions. Malfunction of the exocyst or secretory pathway can lead to dysfunction of the renal system. Disruption in cell to cell contact, an essential barrier to various pathogens, is associated with renal pathological conditions including ischemic acute kidney injury and renal disease (Hsu S, et.al., 1999 Trends Cell Biol 9: 150-153). The exocyst in fully polarized epithelial cells localizes largely, though not exclusively, to the epithelial cell tight junction which acts as a physical barrier between the apical and basolateral plasma membranes.

Sec10 is a central component of the highly conserved eight-protein exocyst complex. Sec10 and Sec15, the most vesicle proximal exocyst components, act as a bridge between the Rab GTPase Sec4/Rab8, found on the surface of the secretory vesicles carrying polarized proteins, and the rest of the exocyst complex that is in contact with the plasma membrane. Perturbation of Sec10 function in mammals has specific and significant inhibitory effects on polarized vesicular delivery. In mammals, overexpression of the N terminal Sec10 subunit acted as a dominant negative and inhibited neurite outgrowth. Sec10 induces cell phenotype changes to taller cells without change of the number of cells per surface area of transwell filter and cell diameter and delivers more E-cadherin into the plasma membrane (Lipschutz J H, et.al., 2000 Molecular Biology of the Cell11: 4259-4275). In the examples below, knockdown of exocyst component Sec10, but not exocyst components Sec8 or Exo70, inhibits ciliogenesis and cystogenesis/ tubulogenesis.

As detailed in the examples, the role of Sec10 in renal tubules of mice following renal ischemia/reperfusion and ROS-damaged cultured tubular epithelial cells was followed. The inventors determined that Sec10-overexpression in vitro leads to increased resistance of tubular epithelial cells against oxidative stress and ERK activation was associated with the resistance. In addition, I/R in mice was associated with exocyst complex. The inventors demonstrated that Sec10-overexpressing cells synthesized more E-Cadherin and delivered more to the basolateral plasma membrane (Lipschutz, 2000, cited above). In collagen matrix 3-dimensional (3D) culture, the Sec10 component of exocyst complex overexpression in MDCK type II cells forms cysts more efficiently and rapidly than in normal MDCK type II cells. These data suggest that Sec10 plays an important role on the intercellular cell to cell contact, including basolateral plasma membrane production.

Further, knockdown of exocyst Sec10 inhibits primary ciliogenesis, cystogenesis, and tubulogenesis, while Sec10 overexpression increases primary ciliogenesis, cystogenesis, and tubulogenesis. The inventors' publication X. Zuo et al, 2009 Mol. Biol. Cell, 20:2522-2529, is incorporated by reference, herein to provide further evidence of this inhibition.

The following examples also demonstrate that Sec10 reduces tubular cell damage caused by hydrogen peroxide due to ERK activation and that Sec10 expression is associated with ischemia and reperfusion injury. To determine if Sec10 was associated with renal epithelial barrier integrity, oxidative stress, and ischemia and reperfusion (I/R) injury, the inventors developed stable Sec10-overexpressing MDCK II cells. The normal MDCK II (wild-type) and Sec10-overexpressing cells grown confluence on the plastic culture dish and formed domes. When cells were treated with hydrogen peroxide, domes were disrupted by the treatment of hydrogen peroxide. The disruption was significantly lower in Sec10-overexpressing cells than in wild-type cells. When cells were grown on the transwell filter, transepithelial electric resistance (TER) of Sec10-overexpressing cells was significantly higher than wild-type cells. Hydrogen peroxide treatment decreased TER. The decrease of TER in Sec10-overexpressed cells was much lower than in control. When cells were grown in the collagen matrix, the cells formed cysts. Hydrogen peroxide damaged the cysts. The damage was significantly lower in Sec10-overexpressed cells than in wild-type cells.

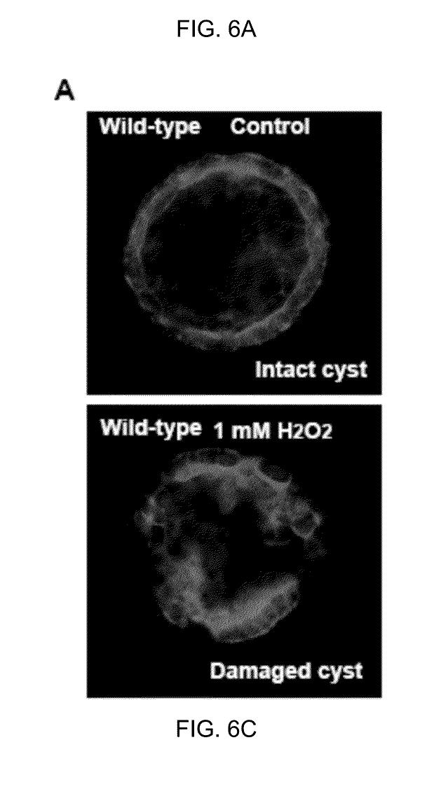

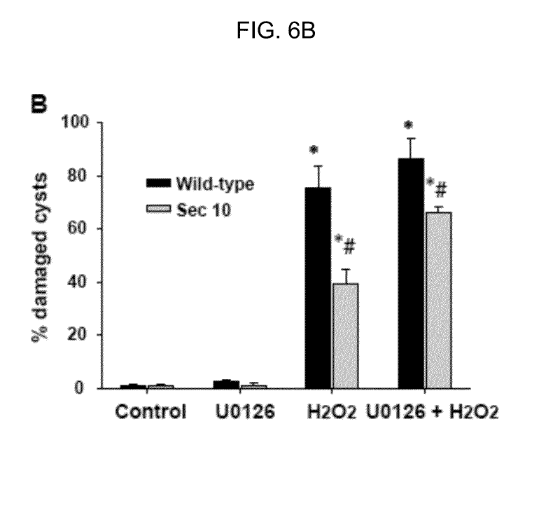

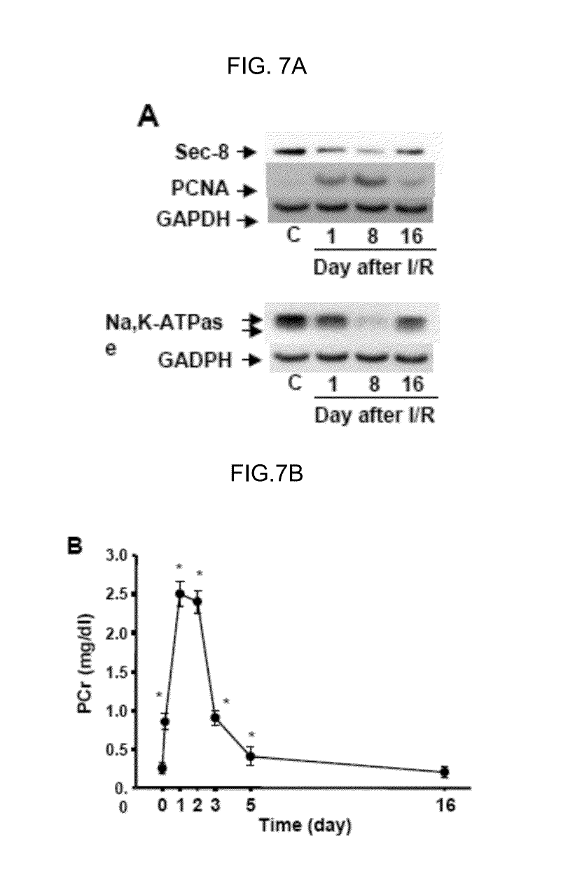

hSec10-overexpression in MDCK cells results in increases of E-Cadherin synthesis and delivery of it to plasma membrane. E-cadherin is localized on adherens junction in both cells. ERK phosphorylation in Sec10-overexpressing cells grown on both plastic culture dish and transwell filter was significantly higher than those in wild-type cells. After treatment of H₂O₂ contact of cell to cell was loosened as seen in FIGS. 6A and 6B. The loss of attachment of intercells was much severe in the control cells when compared with Sec10-overexpressing cells (FIGS. 6A and 6B). Pretreatment with ERK inhibitor, U0126, worsen the loss of tight junction after H₂O₂ treatment in both cells (FIG. 7A, 7B) and exacerbated the decreases of TER induced by hydrogen peroxide and cyst disruption. Exocyst expression in the kidneys subjected to I/R decreased at early after the operation and then gradually returned to normal along with functional recovery. These data support that the higher resistance of Sec10-overexpressing cells against oxidative stress is afforded by the increased delivery of E-Cadherin into junctional areas and that Sec10-overexpression reduced cell damage against oxidative stress via a higher activation of ERK. These data illustrate that an increase of exocyst expression is helpful to accelerate cell recovery or redifferentiation of damaged tubular epithelial cells by increasing stabilization of cell polarity.

According to the methods described herein, the administration of exogenous DNA encoding for Sec10 directly to damaged renal epithelial cells enhances epithelial repair and regeneration and thus recovery from ATN or a related renal tubule or kidney disorders. The overexpression of Sec10 accelerates tubular epithelial cell recovery from ATN. Sec10 is thus useful as a “rescue factor” to speed up recovery for treatment of ATN. Sec10 should similarly protect intact renal tubule epithelial cells when delivered to, and over-expressed in these cells from environmental or genetic damage, when administered prior to the damage.

Thus, for use in the methods and compositions herein, the term “Sec10 nucleic acid” means the nucleotide sequence for human Sec10 identified as GenBank Ref No. NM_{—}006544 (SEQ ID NO: 1). The Sec10 nucleic acids of the invention include the nucleic acid sequence of NM_{—}006544, or fragments thereof of at least 15, at least 50, at least 100, at least 500, at least 1000, at least 3000, at least 5000 or more contiguous nucleotides of the GenBank sequence. A Sec10 nucleic acid sequence also encompasses mutant or variant nucleic acids any of whose bases may be changed from the corresponding base shown in the GenBank reference while still encoding a protein that maintains the Sec10 activities and physiological functions defined herein. A Sec10 nucleic acid sequence or fragment suitable for use in the methods and compositions defined herein include sequences are 100% complementary thereto, including complementary nucleic acid fragments of the lengths defined above. Sec10 nucleic acid sequences or nucleic acid fragments may include chemical modifications, e.g., modified bases to enhance the chemical stability of the modified nucleic acid.

In a similar manner, for use in the methods and compositions herein, the term “Sec10 protein” means the protein sequence identified in GenBank Ref No. NP_{—}006535 (SEQ ID NO: 2), fragments, epitopes or domains thereof, or derivatives, analogs or homologs thereof A Sec10 fragment includes a sequence of at least 15, at least 50, at least 100, at least 200, at least 400, at least 500, at least 700 or more contiguous amino acids of the GenBank sequence. A Sec10 protein includes mutant or variant proteins any of whose residues may be changed from the corresponding residue shown herein while still encoding a protein that maintains the Sec10 activities and physiological functions described herein, or a functional fragment thereof

One of skill in the art may select the appropriate Sec10 sequence based upon the knowledge in the field and the teachings provided herein. See also U.S. Pat. No. 6,964,849 and reference 16.

### C. AAV VECTORS AND COMPOSITIONS USEFUL IN THE METHODS

In certain embodiments of this invention, the Sec10 nucleic acid sequence is delivered to the renal tubule epithelial cells in need of treatment by means of a viral vector or non-viral vector or a plasmid, of which many are known and available in the art. For delivery to the kidney, the therapeutic vector is desirably non-toxic, non-immunogenic, easy to produce, and efficient in protecting and delivering DNA into the target cells. The exogenous Sec10 nucleic acid sequence can be delivered with non-viral or viral vectors. In one particular embodiment, a viral vector is an adeno-associated virus vector.

More than 30 naturally occurring serotypes of AAV are available. Many natural variants in the AAV capsid exist, allowing identification and use of an AAV with properties specifically suited for renal tubular epithelial cells. AAV viruses may be engineered by conventional molecular biology techniques, making it possible to optimize these particles for cell specific delivery of Sec10 nucleic acid sequences, for minimizing immunogenicity, for tuning stability and particle lifetime, for efficient degradation, for accurate delivery to the nucleus, etc.

Thus, Sec10 overexpression can be achieved in the renal tubule epithelial cells through delivery by recombinantly engineered AAVs or artificial AAV's that contain sequences encoding Sec10. The use of AAVs is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector; it has been widely used for efficient gene transfer experiments in different target tissues and animal models. Clinical trials of the experimental application of AAV2 based vectors to some human disease models are in progress, and include such diseases as cystic fibrosis and hemophilia B. Other AAV serotypes include AAV1, AAV3, AAV4, AAV5, AAV6, AAV7, AAV8 and AAV9.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus exemplary AAVs, or artificial AAVs, suitable for expression of Sec10, include AAV2/8 (see U.S. Pat. No. 7,282,199), AAV2/5 (available from the National Institutes of Health), AAV2/9 (International Patent Publication No. WO2005/033321), AAV2/6 (U.S. Pat. No. 6,156,303), and AAV.rh8 (International Patent Publication No. WO02003/042397), among others. A number of these AAVs are used as delivery vectors in the examples provided below.

In one embodiment, the vectors useful in compositions and methods described herein contain, at a minimum, sequences encoding a selected AAV serotype capsid, e.g., an AAV8 capsid, or a fragment thereof. In another embodiment, useful vectors contain, at a minimum, sequences encoding a selected AAV serotype rep protein, e.g., AAV8 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV8 origin. Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199.

The AAV vectors of the invention further contain a minigene comprising a Sec10 nucleic acid sequence as described above which is flanked by AAV 5′ ITR and AAV 3′ ITR.

A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and a Sec10 nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene, i.e., Sec10. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the invention may be delivered to the packaging host cell in the form of any genetic element which transfers the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, 1993 J. Virol., 70:520-532 and U.S. Pat. No. 5,478,745, among others.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

The minigene is composed of, at a minimum, a Sec10 nucleic acid sequence (the transgene) and its regulatory sequences, and 5′ and 3′ AAV inverted terminal repeats (ITRs). In one desirable embodiment, the ITRs of AAV serotype 2 are used. However, ITRs from other suitable serotypes may be selected. It is this minigene which is packaged into a capsid protein and delivered to a selected host cell. The Sec10 nucleic acid coding sequence is operatively linked to regulatory components in a manner which permits transgene transcription, translation, and/or expression in a host cell.

In addition to the major elements identified above for the minigene, the AAV vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1promoter (Invitrogen). Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression. Another embodiment of a regulatory sequence is a tissue-specific promoter.

Suitable regulatory sequences, such as the cytomegalovirus promoter/enhancer, etc. may be selected by one of skill in the art from among many known lists of same. Similarly the methods for assembling and creating recombinant AAV vectors are well-known. Suitable regulatory sequences and methods for assembly and production of an AAV that are useful in this invention include those identified in U.S. Patent No. 7,282,199, incorporated by reference herein.

### D. THERAPEUTIC/PROPHYLACTIC COMPOSITIONS

In one specific embodiment, a therapeutic composition is a useful vector for the methods of this invention is an adeno-associated virus (AAV) comprising an AAV capsid having an amino acid sequence of a AAV2/8 serotype, an amino acid sequence of a functional rep gene of a AAV2/8 serotype, and a minigene having AAV inverted terminal repeats and a human Sec10 gene operatively linked to regulatory sequences that direct expression of Sec10 in a human cell. One of skill in the art according to the teachings herein may readily select and assemble other suitable AAV vectors expressing Sec10 from the above components for use for in vitro, ex vivo or in vivo gene delivery to the kidney tubule epithelial cells.

Compositions of this invention therefore include a therapeutic composition comprising an adeno-associated virus (AAV) comprising an AAV capsid having an amino acid sequence of an AAV described above, e.g., AAV2/8 serotype, an amino acid sequence of a functional rep gene of an AAV described above, e.g., AAV2/8 serotype, and a minigene having AAV inverted terminal repeats and a human Sec10 gene operatively linked to regulatory sequences that direct expression of Sec10 in a human cell, in a physiologically compatible carrier. The rAAV, is in one embodiment, suspended in a physiologically compatible carrier, for administration to a human or non-human mammalian patient. Suitable carriers may be readily selected by one of skill in the art. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Dosages of the viral vector will depend primarily on factors such as the condition being treated, e.g., AKI/ATN or pre-MRI testing, or other prophylactic use, the age, weight and health of the patient, and may thus vary among patients. For example, a therapeutically effective human dosage of the viral vector is generally in the range of from about 1 ml to about 100 ml of solution containing concentrations of from about 1×10⁹ to 1×10¹⁶ genomes of virus vector. A preferred human dosage may be about 1×10¹³ to 1×10¹⁶ AAV genomes. The dosage will be adjusted to balance the therapeutic benefit against any side effects and such dosages may vary. The levels of expression of the Sec10 transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

### E. METHOD OF DELIVERY

Performance of the methods described herein involves delivering the desired vector carrying the Sec10 gene in sufficient amounts to transfect the renal tubule kidney cells and to provide sufficient levels of gene transfer and expression to provide the therapeutic or prophylactic benefit without undue adverse effects, or with medically acceptable physiological effects. Such a balancing of desired medically acceptable effects with any adverse side effects can be determined by those skilled in the medical arts.

The kidney is directly accessible for gene delivery by a variety of different routes including renal artery injection, direct injection into the parenchyma, and retrograde injection via the ureter. Other conventional and pharmaceutically acceptable routes of administration include, which would be indirect, include oral, inhalation, intranasal, intratracheal, intraocular, intravenous, intramuscular, subcutaneous, intradermal, and other parental routes of administration. Routes of administration may be combined, if desired. However, for purposes of the methods described herein, in one embodiment, the preferred method of delivery of the vector carrying the Sec10 gene is by retrograde injection into the ureter. See, e.g, the description of this method in the following examples.

Retrograde injection is an attractive route for treating ATN with the AAV carrying Sec10 because as renal tubule cells are the cells affected in AKI/ATN, and these cells are directly accessible by retrograde injection. This mode of administration allows an effective dose to be determined and administered, without concern about any substantial distribution to and through other organs of the body. The use of retro-ureteral injection as the route of AAV delivery permits the Sec10 to be expressed at the site of renal epithelial cell damage and does not permit the exogenous DNA to substantially reach the blood stream. This permits directed therapy and lowers the risk of immune reaction to any components of the therapeutic composition. Retrograde injection into kidneys will reduce or eliminate contact with the bloodstream, thereby reducing chances of immune-related side effects. Furthermore, the route of delivery, retro-ureteral injection, limits the possibility of toxic or adverse immunologic reactions, as the genetic material, carriers and other components of the composition are not exposed to the bloodstream.

Thus, therapeutic and prophylactic methods and compositions are described that enhance repair of the structure and function of the tubular epithelia damaged in ALI/ATN or other causes of kidney damage by overexpressing Sec10 at the site of renal epithelial cell damage. In the embodiment that employs rAAV as the delivery vehicle via retrograde trans-ureter injection, this treatment is further characterized as safe, non-toxic and non-invasive. As such, these methods and compositions are suitable for therapy of subjects with disease as well for prophylactic use in individuals at risk for developing AKI/ATN or damage kidney tubule epithelial cells in response to environmental causes.

### F. EXAMPLES

The examples that follow do not limit the scope of the embodiments described herein. In the following examples, inventors have demonstrated the protective effect of Sec10 overexpression in MDCK cells exposed to toxic hydrogen peroxide treatment, a chemical mimic for ischemic ATN. When normal MDCK Type II (control) and Sec10-overexpressing cells were grown to confluence on plastic culture dishes, they formed typical domes. When MDCK cells were treated with hydrogen peroxide, an in vitro model of I/R injury involving oxidative stress, domes were disrupted. The disruption was significantly less in Sec10-overexpressing cells, compared to control MDCK cells. When cells were grown on Transwell filters, transepithelial electric resistance (TER) in Sec10-overexpressing cells was significantly higher than in control MDCK cells. Hydrogen peroxide treatment decreased TER in all cells, but the decrement in TER in Sec10-overexpressing cells was significantly less than in control cells. When the cells were grown in a three-dimensional (3D) Type I collagen matrix, they underwent epithelial morphogenesis and formed typical cysts. Hydrogen peroxide treatment damaged the cysts, and the damage again was significantly less in Sec10-overexpressing cells versus control cells. The mitogen activated protein kinase (MAPK) pathway has been shown to protect animals from I/R injury. Levels of active (phosphorylated) extracellular signal-regulated kinase (ERK), the final protein in the MAPK pathway, were higher in Sec10-overexpressing compared to control cells grown on both plastic culture dishes and Transwell filters. U0126, an inhibitor of ERK activation, exacerbated both the decreases of TER and cyst disruption induced by hydrogen peroxide.

The ability of various AAV serotypes to efficiently transduce reporter genes in the MDCK cell line and in the tubular epithelia of the mouse kidney is shown. Preliminary data show the feasibility of transducing renal tubular cells via the retrograde route and obtaining rapid onset transgene expression with novel AAVs carrying reporter genes. The examples demonstrate that knockdown of Sec10 inhibits cystogenesis/tubulogenesis. The examples used a mouse model of ATN, in which ischemia is induced by cross-clamping the renal artery followed by release of the clamp and subsequent reperfusion (Dobashi K, et. al., 2000 Mol Cell Biochem 205: 1-11, 2000; Finger F P, et. al., 1998 Cell 92: 559-571).

Transduction of the mouse kidney was done through the retro-ureteral delivery method. In mice subjected to renal I/R injury, exocyst expression levels decreased early after induction and gradually returned to normal along with functional recovery. The examples provide evidence that the exocyst, via Sec10 expression, is involved in the recovery following and or resistance to I/R injury. One skilled in the art will appreciate that modifications can be made in the following examples which are intended to be encompassed by the spirit and scope of the invention.

### Example 1

### The MAPK Pathway is Centrally Involved in MDCK Tubulogenesis In Vitro MDCK Cell System

Due to the complexity of organogenesis (the kidney is composed of more than twenty cell types and one million nephrons) and the transitory nature of cyst and tubule formation, it is difficult to study these processes in vivo. Relatively little, therefore, was known about cyst and tubule formation prior to development of an in vitro assay. The Madin-Darby canine kidney (MDCK) cell line, derived from the kidney tubules of a normal cocker spaniel in 1958, has been one of the most widely used systems for studying fundamental issues in epithelial cell biology. It was first observed that MDCK cells seeded to plasma fibrin, or collagen-coated sponge, formed multicellular structures. When MDCK cells were seeded within a three-dimensional collagen matrix, over ten to fifteen days they formed structures which were characterized by a polarized epithelium surrounding a fluid-filled space, apical microvilli, a solitary cilium, and apical tight junctions , meeting the most rigorous definition of “cysts”. Following induction of the MDCK cell cysts with hepatocyte growth factor, tubulogenesis occurred. The utility of such three dimensional culture systems have been greatly increased by confocal microscopy, which permits the facile visualization of cystogenesis/tubulogenesis and immunocytochemical localization of proteins .

The MAPK pathway regulates MDCK tubulogenesis in vitro. Using MDCK cells that were grown in a collagen matrix to the cyst stage and then induced to undergo tubulogenesis with HGF, tubulogenesis is divided into two stages, the partial epithelial to mesenchymal transformation (p-EMT), dependent on the MAPK pathway, and redifferentiation, which was dependent on matrix metalloproteinases (MMPs). Using a canine DNA microarray, several candidate proteins were identified as having involvement in the p-EMT stage of tubulogenesis, including Claudin 2 and Fibronectin. Both of these proteins are centrally involved in p-EMT and activated by the MAPK pathway. As further confirmation of the importance of the MAPK pathway in p-EMT, a strain of MDCK cells, Type I, was used that were of collecting duct origin and had high levels of active ERK (the final phosphorylated target of the MAPK pathway). These cells were determined to spontaneously initiate tubulogenesis. Blocking activation (phosphorylation) of ERK, prevented tubulogenesis. Ureteric bud cells, the precursor of collecting ducts, were then examined and found to have high levels of active ERK and spontaneously initiate tubulogenesis. ERK inhibition prevented tubulogenesis. Previously, in kidney explants in organ culture, preventing ERK phosphorylation/activation resulted in an inhibition of branching morphogenesis and development. A second microarray and a technique termed “subtraction pathway microarray analysis” were used to identify the specific MMPs and tissue inhibitors of matrix metalloproteinases (TIMPs) involved in the redifferentiation of tubulogenesis. After identifying MMP-13 TIMP1 as candidates, shRNA was used to knockdown MMP13 and TIMP1, and showed that these proteins were both necessary for the redifferentiation stage of tubulogenesis and regulated by the MAPK pathway. Activation of the MAPK pathway attenuated tubular cell injury following ischemia/reperfusion in vivo.

### Example 2

### Materials and Methods

A. Cell Culture

Type II Madin-Darby canine kidney (MDCK) cells (Control cells) were obtained from Dr. K. Mostov (UCSF, San Francisco, Calif.). MDCK type II cells were overexpressing hSec10 (Sec10-overexpressing cells). See, e.g, Lipschutz et al, 2000 cited above. Cells were grown in modified Eagle's minimal essential medium (MEM) containing Earl's balanced salt solution and glutamine supplemented with 5% fetal calf serum, 100 U/ml penicillin, and 100 μg/m streptomycin on the plastic culture dishes. Some cells were grown on the 24-mm Transwell 0.45 μm polycarbonate filter units coated with collagen (Corning Life Sciences, Lowell, Mass.). Pore size on all filters was 0.4 μm. Cell monolayers were used for experiments after 7 d of culture with daily changes in medium. Cells were plated as single cells in a three-dimensional (3D) type I collagen gel. To culture collagen matrix, cells grown on plastic culture dishes were harvested using trypsin-EDTA, and suspended cells on the type I collagen gel and then seeded in chamber slide for 14 days and then treated with H₂O₂ (Sigma-Aldrich, Co.).

B. Measurement of TER

Control cells and Sec10-overexpressing cells were grown on the Transwell filter for 7 days after seeding. TER was measured using epithelial volt-ohmmeter (Model EVOM, World Precision Instruments). TER values were presented as the measured resistance in ohm multiplied by the surface area of the Transwell filter.

C. Mouse Ischemia and Reperfusion

Experiments were performed in 8 week old C57BL/6 mice. Mice were allowed free access to water and standard mouse chow. In all cases, studies were conducted according to the animal experimental procedures approved by the Kyungpook National University Institution Animal Care and Use Committee. Kidney ischemia was carried out using known procedures. Briefly, animals were anesthetized with pentobarbital sodium (60 mg/kg body weight, BW; ip) prior to surgery Animals were subjected to either 30 min of bilateral renal ischemia or sham operation on 0 day. Body temperature was maintained at 36.6-37.5° C. throughout the procedure. To induce ischemia/reperfusion, renal pedicles were occluded using nontraumatic microaneurism clamps (Roboz, Rockville, Md.) which were removed after 30 min. Kidneys were snap-frozen for biochemical studies. Each animal group consisted of more than four mice.

D. Renal Functional Parameters

To evaluate concentration of plasma creatinine (PCr), 70 μl of blood was taken from the orbital sinus at the indicated time in FIG. 7A-7B (n=4). PCr concentration was measured using the Beckman Creatinine Analyzer II (Beckman, Brea, Calif.).

E. Western Blot Analysis

Briefly, cells or kidneys were harvested in the RIPA (Sigma Co.) containing proteinase inhibitor cocktail (Sigma. Co) and phosphatase inhibitor cocktail (Sigma. Co) and centrifuged 14,000 rpm for 20 min at 4° C. Supernatants, then, were collected and protein concentration was determined using BCA protein assay kit. Protein samples were mix SDS-sample buffer and denatured by 5 min of boiling at 95° C. The protein samples were separated on 4-12% SDS-PAGE gels and then transferred to an Immobilon membrane (Millipore Corp., Bedford, Mass.). The membranes were blocked by 5% of non-fat dry milk in PBS containing of 0.1% Tween-20 (PBS-T), and incubated in anti-phospho-ERK (1:1000, Cell signaling), -total-ERK (1:5000, Cell signaling), and -Sec10 (1:500; Lipschutz lab.) antibodies overnight at 4° C. After washing with 3 times with 0.1% PBS-T, the membranes were incubated with horseradish peroxidase-conjugated secondary antibodies for 1 h at RT. Finally, the membranes were exposed to a Western Lighting Chemiluminescence Reagent (Perce).

F. Immunofluoresence Staining

Cells were grown in collagen gel and fixed with 4% paraformaldehyde for 30 min at 4° C. after digesting in collagenase (100 U/ml; Sigma-Aldrich, St. Louis, Mo.) for 10 min at 37° C. The cyst was permeablized with 0.025% saponin in PBS containing 0.7% fish skin gelatin (PFS buffer) for 30 min at room temperature. The collagen gels were blocked and incubated in phalloidin-rodamine for 2 hrs at room temperature, washed and mounted with mounting medium (Vectershield). Antibodies were diluted in PFS buffer.

G. Statistics

Results were expressed as mean±SEM. Statistical differences among groups were calculated using analysis of variance (ANOVA) followed by a least significant difference post hoc comparison using the SPSS 12.0 program. Differences between groups were considered statistically significant at a P value of <0.05.

### Example 3

### The Exocyst Relocalizes During Tubulogenesis Consistent with a Role in Directing Membrane Traffic

Localization and relocalization of a protein complex are suggestive of function. As revealed in photographs (not shown), the exocyst was found to relocalize during the various stages of cyst and tubule formation, coincident with changes in cell polarity (Guo W, et.al., 1999 EMBO J 18: 1071-1080). In MDCK cells grown for ten days in a collagen gel, a fluid-filled cyst is formed in which staining is seen at the area of the tight junction using anti-exocyst Sec8 antibody. In a similar fluid-filled cyst formed by MDCK cells grown for ten days in collagen and stimulated with HGF, the exocyst can be seen relocalizing along the growing tubules in a pattern consistent with the changes in polarity that occur as tubules form. Sec8 is seen relocalizing into the extension. In another photograph of the cysts described immediately above, but during the cord stage of tubulogenesis, staining occurs at the region of cell-cell contact in the cord. This region becomes the boundary of a new lumen. Yet another photograph of the same cysts shows a nascent tubule in the final stage of tubulogenesis, with two vertical lines of Sec8 staining outlining the boundary of the lumen.

This evidence shows that exocyst overexpression leads to increased cyst and tubule formation. During HGF-induced formation of tubules from MDCK cells grown as cysts, the cells go through a dramatic sequence of changes in polarity and shape. The relocalization of the exocyst during tubulogenesis is highly suggestive of the redirection of delivery of new membrane and secretory products to the growing extensions and tubules during the physiologic remodeling of cell shape and polarity that occurs throughout the tubulogenic process. This relocalization is strikingly similar to the way in which the exocyst is involved in redirecting vesicles, carrying polarized proteins, to different regions of the plasma membrane during the yeast life cycle (Thadhani R, et.al., 1996 New Engl. J. Medic. 334: 1448-1460).

To show causality of function, a crucial exocyst component, the vesicle-proximal Sec10 was overexpressed during growth of MDCK cells in a collagen matrix. In the hSEC10 over-expressing MDCK cells grown in collagen matrix and induced with HGF, photomicrographic evidence (confocal microscopy, with actin staining; not shown) demonstrated that Sec10 overexpression resulted in increased cyst and tubule formation, indicating that the exocyst was centrally involved in both cystogenesis and tubulogenesis.

After 7 days of growth in a collagen matrix, cysts composed of human Sec10 (hSec10)-overexpressing MDCK cells were often mature. After 7 days of growth in a collagen matrix, control cell (non-hSec10-expressing) cysts were still incompletely formed. Nomarski imaging of mature human Sec10-expressing cysts grown for ten days in collagen with HGF-induced stimulation revealed a number of tubules in the hSec10-overexpressing cell cysts. In another photograph employing Nomarski imaging of mature control cysts, a lesser number of tubules than in the hSec10-overexpressing cell cysts was revealed. (Figures not shown).

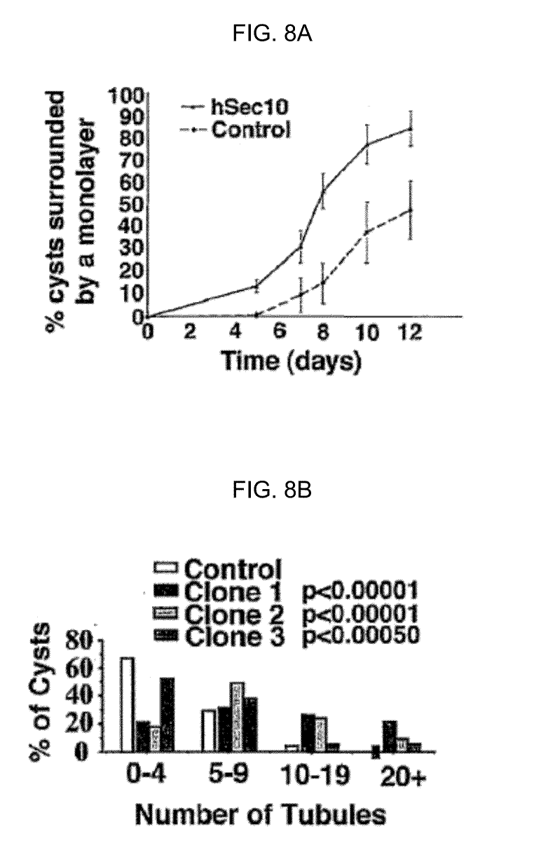

FIGS. 8A and 8B illustrate the increased rate and efficiency of mature cyst formation in the overexpressing cells and the number of tubules per cyst of the cultures described above, respectively.

### Example 4

### Exocyst Expression Increases as Tubulogenesis Begins During Kidney Development In Vivo

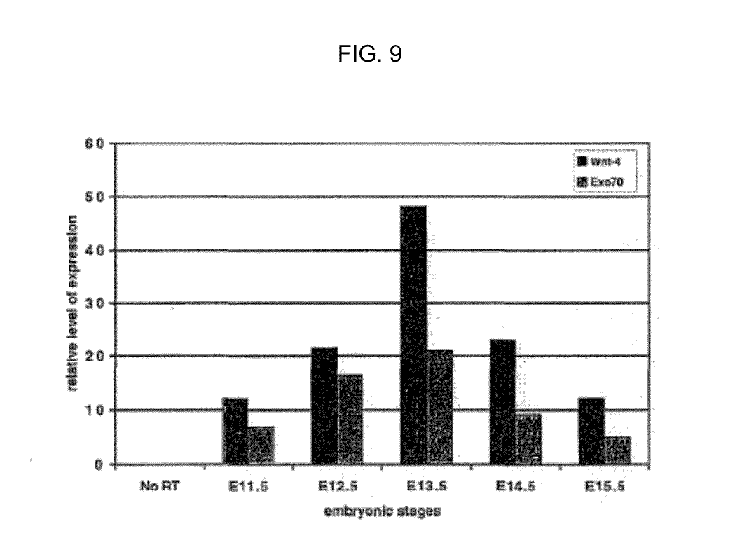

Murine kidneys were harvested at various embryonic stages and RNA was obtained. cDNA was generated by reverse transcription, followed by real-time PCR. Exocyst expression occurred at embryonic day 11.5 (E11.5), increased three-fold by E13.5, and then decreased by E1 5.5 to levels at or below those seen at E11.5. Wnt-4 followed a similar pattern and has been shown, by knockout models, to be involved in the mesenchymal epithelial transformation that denotes the beginning of tubulogenesis. See FIG. 9.

### Example 5

### Sec10-Overexpressing Cells are Resistant to Oxidative Stress and Have Increased Active (Phosphorylated) ERK Levels

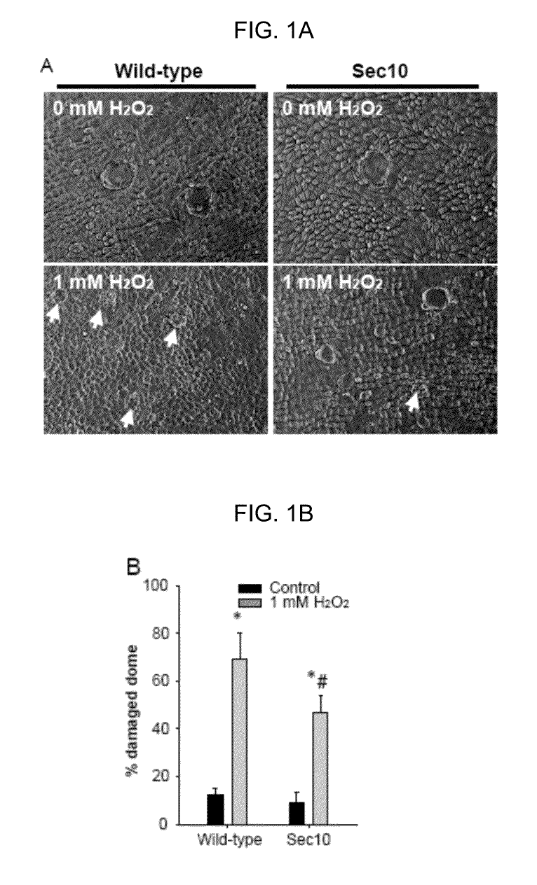

Human Sec10-overexpressing type II MDCK cells, which present taller phenotype, were used, as described in Lipschutz et al 2000 cited above. Control (wild-type) and hSec10-overexpressing (Sec10) MDCK cells were grown on plastic culture dish to the point of confluence and formation of dome (see arrows in FIG. 1A). The confluent grown cells were treated with no (0) or 1 mM H₂O₂ (which is an in vitro model of ischemia/reperfusion (I/R) injury involving oxidative stress) for 30 minutes.

Confluent cells grown on plastic culture dishes with no hydrogen peroxide formed dome due to active secretion by the MDCK cells and tight cell to cell contact which could be enough to lift the cells off the plastic culture dish. Evidence of the involvement of Sec10 on integrity of cell to cell contact, was revealed when control and Sec10-overexpressing cells forming domes were treated with 1 mM H₂O₂.

Sec10-overexpressing cells were resistant to treatment with H₂O₂, while control MDCK cells were damaged, as evidenced by the inability to form a tight monolayer and domed structures. Control MDCK cells no longer had the typical MDCK “cobblestone” appearance and were unable to form domes. FIG. lA are photographs of these cells taken with Olympus light microscope showing that SEC10-overexpression resulted in reduced loss of dome. Domes are due to active secretion by the MDCK cells that lifts the cells off the plastic culture dish. Arrows indicate damaged domes. H₂O₂ treatment disrupted domes (FIG. 1A, arrow). FIG. 1B plots the numbers of damaged and intact domes from these cells. The disruption was significantly lower in Sec10-overexpressing cells than control cells, providing evidence that Sec10 is associated with cell to cell contact.

To determine if the ERK signal pathway involves the higher resistance of Sec10-overexpressing cells to H₂O₂ insult, levels of active ERK in control and Sec10-overexpressing cells grown on plastic culture dishes and in transwell filter were determined.

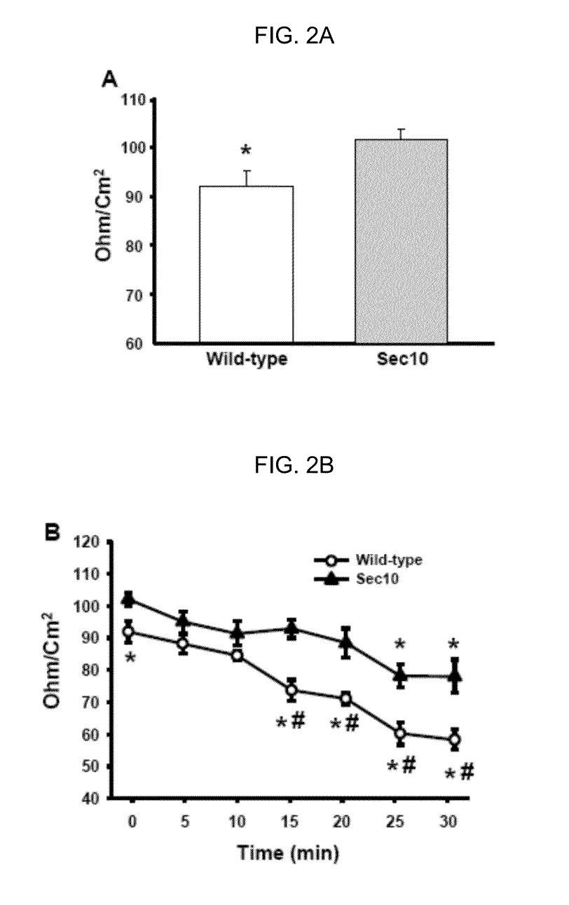

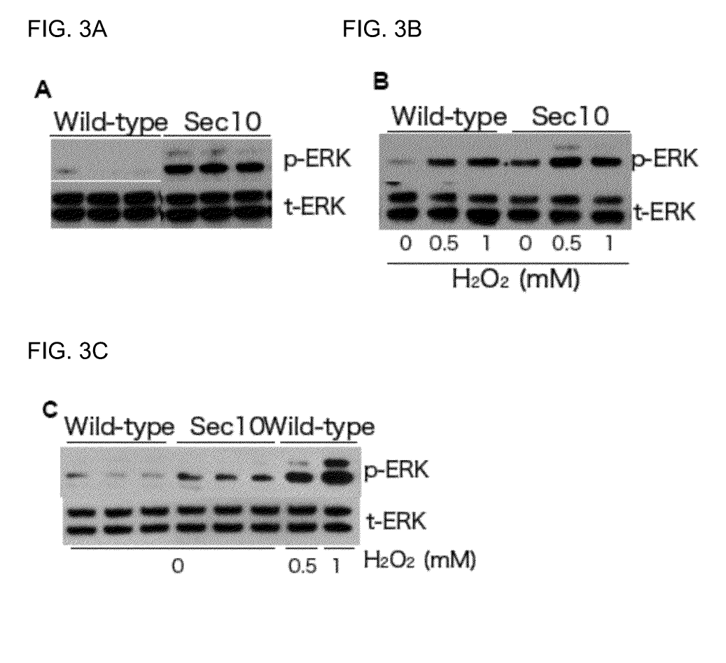

Phosphorylated (active) ERK levels in Sec10-overexpressing cells grown on the plastic culture dishes were significantly higher than in control cells, which can be seen by comparing FIG. 3B with FIG. 3A. Consistent with results of the plastic culture dish, in collagen matrix, active ERK levels were higher in Sec10-overexpressing cells than in normal cells (FIG. 3C). Hydrogen peroxide treatment resulted in ERK phosphorylation (See, FIGS. 2B and 2C).

Sec10-overexpressing cells, which re resistant to oxidative stress, were found to have increased levels of phosphorylated (active) ERK, which likely explains the increase in tubulogenesis seen in Sec10 overexpressing cells grown in type-1 collagen and induced with HGF described in Example 3. ERK activation is involved in TER and cell damage induced I/R insult and oxidative stress. Sec10-overexpression was found to exacerbate morphogenesis which is regulated by ERK activation.

### Example 6

### Increased In Vivo Exocyst Expression in Recovering Kidneys Following Ischemia/Reperfusion

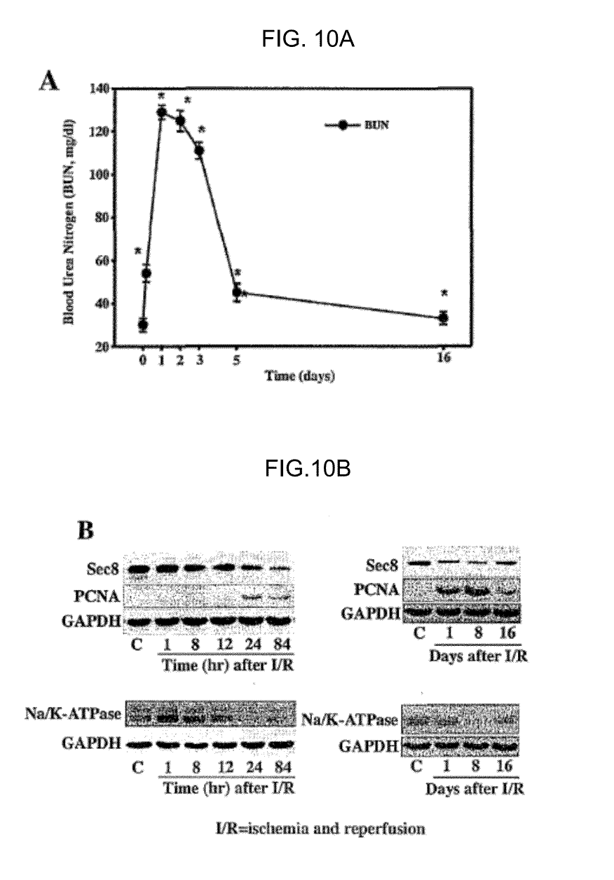

Markedly increased tubulogenesis followed treatment with HGF of Sec10 overexpressing, compared to control, cell cysts, as described in Example 3 and in FIGS. 8A-8B. For in vivo correlation of these results, exocyst expression in kidneys recovering from ATN was examined. Using the murine ischemia/reperfusion model, exocyst expression in kidneys was investigated following ischemia and reperfusion. Exocyst expression first decreased and then increased coincident with renal tubule recovery and redifferentiation, providing evidence to support the involvement of the exocyst in tubular recovery following ATN (FIGS. 10A-10B).

### Example 7

### AAV Efficiently Transduces Renal Collecting Duct Cells In Vitro

MDCK Strain I cells were cultured as confluent epithelial monolayers on Transwell-Clear membranes and exposed to AAV2/5 carrying wild-type (wt) or mutant (mt) FLAG-tagged EGF containing fibrillin-like extracellular matrix protein 1 (EFEMP1). The viral constructs were AAV2/5.EFEMP1-wt or AAV2/5.EFEMP1-mt. AAV2/5 encoding enhanced green fluorescent protein (EGFP) alone was used as a non-secreted control. Western gels showed directional (apical) secretion of both wild-type and mutant EFEMP1 as observed through immunoprecipitation of basal and apical media at 24, 48 and 72 hours post-infection (gels not shown). Apical secretion persisted through 72 hours after infection (the latest timepoint evaluated). As anticipated, EGFP, delivered by infection of additional aliquots of cells via AAV2/5.EGFP, was not observed in the media.

This experiment showed that AAV2/5 (i.e., AAV2/5.EFEMP1-FLAG) efficiently transduces polarized MDCK Strain I cells, which are of collecting duct origin, in vitro and transgenic proteins undergo the predicted cellular processing. This is the technique that was used to identify the AAV serotype that most efficiently infects collecting duct cells in vitro.

### Example 8

### Retrograde Delivery of AAV

Briefly, the mice were anesthetized and the left kidney exposed via a 2 cm flank incision. A clamp was placed on the ureter below the injection site to prevent leakage to the bladder. Using a 3D-gauge needle and a microinjection apparatus, AAV particles were injected into the ureter just below the ureteropelvic junction. The total volume of viral solution ranges from 50-100 μl. After 5-15 minutes, the clamp was removed and the site was surgically closed.

### Example 9

### AAV Allows for Protein Expression in the Kidney

The use of AAV-mediated gene transfer was successfully tested in the renal collecting system. For these studies, 10⁹ genome copies of selected AAV serotypes carrying minigenes containing either green fluorescent protein or luciferase under the control of the CMV promoter, CMV.EGFP or CMV.Luciferase (provided by the University of Pennsylvania Vectorcore) were delivered via retrograde injection into the kidneys of wild-type mice. These vectors were prepared using methods such as described in U.S. Pat. No. 7,282,199, among others.

Two to three weeks after these injections, animals were imaged for luciferase activity or kidneys were harvested and evaluated for presence of EGFP. In one mouse, imaging of luciferase bioluminescence was taken using the Xenogen IVIS system 2 weeks after retrograde injection of AAV2IB.CMV.Luciferase to the left ureter (photo not shown). EGFP fluorescence in urine kidneys dissected from different animals 3 weeks after delivery of the designated AAV carrying CMV.EGFP to the ureter were also photographed (not shown). In these figures, AAV2/8 and AAV2/9 serotypes resulted in high levels of reporter gene expression specific to the targeted kidney; lower levels of transgene expression were detected after injection of AAV2/6 and AAV.rh8.

Histological studies revealed that EGFP was efficiently and specifically expressed in high levels in renal tubular epithelial cells in the kidney region (medulla) exposed to the virus and in the cortex after retro-ureteral delivery of AAV.EGFP, but not in the contralateral untreated kidney (figure not shown). There was no evidence of an inflammatory/immune response relating to presence of AAV capsid antigens or the reporter protein.

While a number of recombinant viruses, including lentivirus, adenovirus, and AAV serotypes 1-5 have been tested in vivo in the kidney, none have resulted in as efficient or as stable transduction of tubular epithelial cells as we observed with the novel viruses AAV2/8 and AAV2/9. These results provide evidence that wild-type Sec10 can be efficiently delivered and expressed in renal tubule cells.

### Example 10

### Sec10-Overexpression Inhibits the Decrease of Transepithelial Electric Resistance (TER) Caused by Treatment of Hydrogen Peroxide

TER is a sensitive parameter to determine the integrity of cell to cell contact, which is highly associated with various kidney diseases (Welsh M J, et.al., 1985 J. Clin. Invest. 76: 1155-1168). TER of Sec10-overexpressing cells was significantly higher than that of control cells (FIG. 2A). Hydrogen peroxide resulted in decrease of TER over time (FIG. 2B). The TER decrease by hydrogen peroxide was significantly higher in the control cells than in Sec10-overexpressing cells (FIG. 2B). These data indicate that Sec10-expression involves in cell to cell contact and cellular permeability in the kidney epithelial cells.

TER is known to be a sensitive measure of barrier function and integrity of tight junctions. Grown cells on the transwell increased gradually TER overtime and the TER were not significantly changed 5 days after seeding the cells. After confluent growing, TER in Sec10-overexpressing cells was higher than in control cells. These data suggest that Sec10 overexpression cells may develop more integrated tight junction and cell adherence proteins. Sec10-overexpression changes cell phenotype to taller and larger plasma membrane surface. It may be an explanation of the higher TER. In addition, Sec10-overexpression may increase the expression and stability of attachment to cytoskeleton proteins of tight junction such as ZO-1. As shown in the examples, the inventors found slightly higher ZO-1 expression in Sec10-overexpressing cells. Nevertheless differences of ZO-1 protein amount between these cells were statistically significant.

### Example 11

### ERK Inhibition Exacerbates Decrease of Transepithelial Electric Resistance Caused by Hydrogen Peroxide Treatment

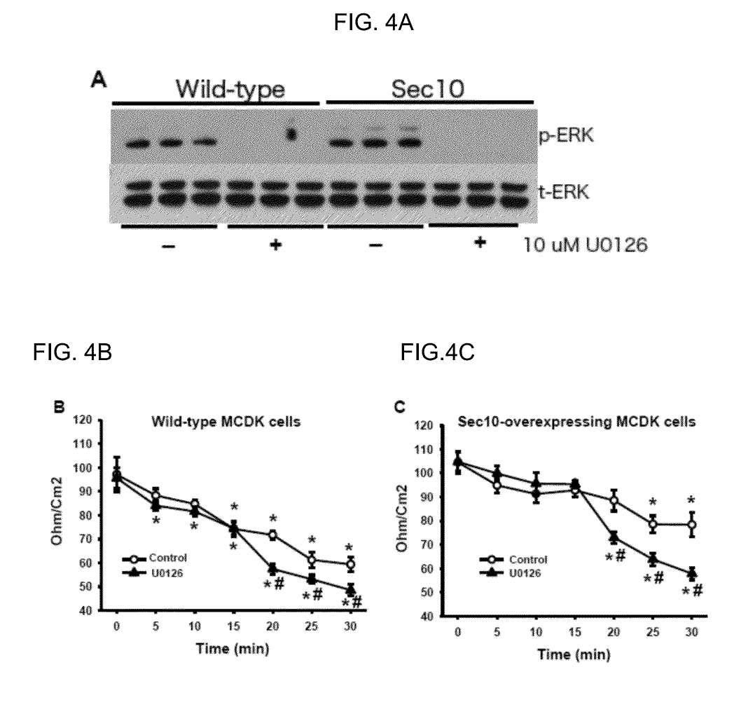

To investigate whether the highly activated ERK in Sec10-overexpressing cells contributes to the resistance to H₂O₂ treatment, ERK activation of Sec10-overexpressing cells was blocked using U0126, a specific ERK inhibitor and then cells were treated with H₂O₂. As seen in FIG. 4A, UO126 treatment blocked ERK phosphorylation caused by H₂O₂ treatment. Pretreatment of U0126 for 30 min accelerated H₂O₂-induced decrease of TER in both control (FIG. 4A) and Sec10-overexpressing cells (FIG. 4B).

To investigate the involvement of Sec10 and ERK activation on recovery of TER after H₂O₂ treatment, two experiments were carried out:

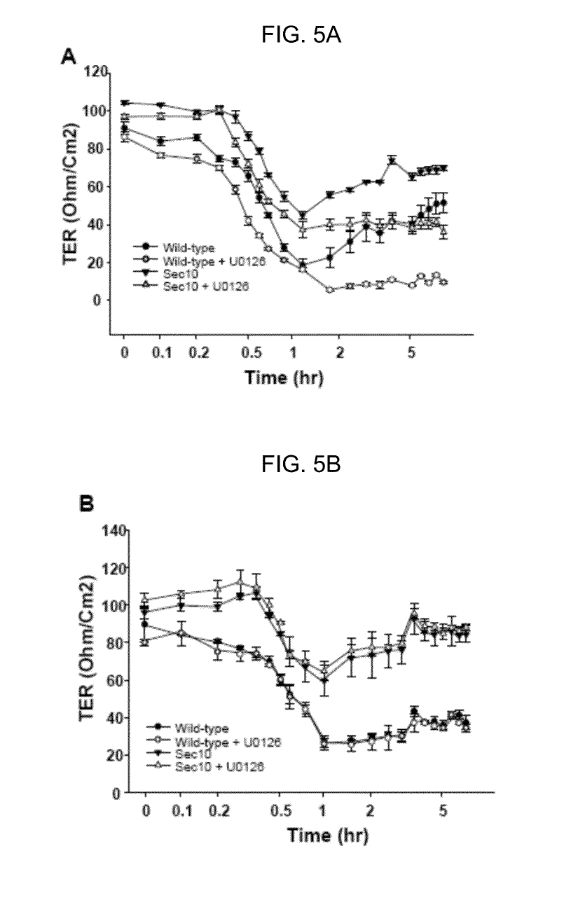

1) control and Sec10-overexpressing cells were treated with U0126 alone for 30 min, U0126 plus H₂O₂ for 30 min, and then U0126 alone for 24 hrs (FIG. 5A), and

2) control and Sec10-overexpressing cells were treated with H₂O₂ for 30 min, and U0126 alone for 24 hrs (FIG. 5B).

Consistent with FIG. 4B and C, pretreatment of U0126 accelerated the decrease of TER induced by H₂O₂ in both control and Sec10-overexpressing cells (FIG. 5A). The decreased TERs were not recovered after change with both normal MEM medium and medium containing U0126 in both control and Sec10-overexpressing cells until 6 hrs after removal of H₂O₂ (FIG. 5A). Twenty-four hrs after removing of H₂O₂ TER was significantly than H₂O₂-untreated levels. The recovery in control cells treated with U0126 was not significantly different as compared with non-U0126 treated cells (FIG. 5B). However TER in U0126-treated Sec10-overexpressing cells was significantly lower than that in U0126-nontreated Sec10-overexpressing cells. This result indicates that ERK activation is associated with TER recovery (FIG. 5B). Nevertheless TER decreases were lower in Sec10-overexpressing cells when compared with control cells. TER recovery in Sec10-overexpressiong U0126-treated cells was not faster than in control cells (FIG. 5B). TER recovery in Sec10-overexpressing cells is more dependent on the ERK activation when compared with control cells.

To clarify carefully whether the recovery is involved in ERK activation, cells were treated first with H₂O₂ for 30 min and then U0126 without H₂O₂ for 24 hrs. Until 6 hrs after U0126 treatment levels of TER were no significant difference between experiment group, but 24 hrs after treatment TER in control cells-treated with H₂O₂ and following U0126 was about 83% of TER in H₂O₂ alone, indicating that ERK inhibition delayed TER recovery (FIG. 5D). Similar to control cell results, TER in Sec10-overexpressing cells treated with H₂O₂ and following U0126 treatment was about 80% of the TER in Sec10-overexpressing cells treated with H₂O₂ alone at 24 hrs after treatment (FIG. 5D). Because damage levels were not same between control and Sec-10 overexpression (FIG. 5B), the TER result which was measured 24 hrs after H₂O₂ removal clearly showed ERK activation is involved in the recovery of TER.

### Example 12

### Sec10-Overexpression Prevents Cysts Against Hydrogen Peroxide

MDCK cells form cysts in the 3 dimensional (3D) collagen matrix. MDCK cells grown in the 3D collagen matrix formed cysts (FIG. 6A). H₂O₂ treatment resulted in damaged cysts (FIG. 6C) as reflected by the change of phalloidin localization and staining intensity. Phalloidnin localized strongly apical and basement membrane before H₂O₂ treatment (FIG. 6A). When damaged cyst numbers were counted, the numbers of damaged cysts (FIG. 6C) in control MDCK cells were significantly higher than in Sec10-overexpressing cells (FIG. 6B). To investigate the role of ERK activation in the cyst damages, cells were incubated in the medium containing U0126 for 30 min and then treated with 1 mM of H₂O₂ plus U0126 for 30 min. Numbers of damaged cysts increased by U0126 treatment in both normal and Sec10-overexpressing cells was shown in FIG. 6B.

### Example 13

### Transient Ischemia and Reperfusion Changes Exocyst Expression in the Kidneys of Mice

To investigate a correlation of exocyst expression and damage in the in vivo acute tubular injury animal model, exocyst Sec8 expression was determined in kidneys subjected to 30 min of ischemia. Exocyst expression decreased early after reperfusion and increased overtime (FIG. 7A), suggesting that exocyst may contribute to the kidney cell damage and recovery after I/R insult. Expression of PCNA started 1 day after reperfusion, peaked at 8 day, and then decreased (FIG. 7A). The early increase of PCNA seen in 24 hr after reperfusion may be associated with the kidney cell repair, since PCNA expression causes an increase in both proliferative cells and damaged cells to repair the damaged cells. 16 days after reperfusion, PCNA expression was lower than 8 days later, suggesting that the periods of time may be redifferentiation periods. Renal function was dramatically decreased early after I/R injury and then recovered over time (FIG. 7B). Exocyst expression decreased early after reperfusion and then increased coincident with renal tubule recovery and redifferentiation. This data provides evidence that exocysts are associated with tubule cell damage, recovery and redifferentiation. Na,K-ATPase expression showed similar results on exocyst expression (FIG. 7A).

All documents listed in this specification, including the entirety of U.S. provisional patent application No. 61/247,746, and the sequence listing, are incorporated herein by reference. While various embodiments in the specification or claims are presented using “comprising” language, under various circumstances, a related embodiment may also be described using “consisting of” or “consisting essentially of” language. It is to be noted that the term “a” or “an”, refers to one or more, for example, “a reagent,” is understood to represent one or more reagents. As such, the terms “a” (or “an”), “one or more,” and “at least one” are used interchangeably herein. While the invention has been described with reference to specific embodiments, it is appreciated that modifications can be made without departing from the spirit of the invention. Such modifications are intended to fall within the scope of the appended claims.

1. A method for treating a mammalian subject having damage to the tubular epithelial cells, the method comprising:
delivering to the tubular epithelial cells via retrograde injection into the ureter of the subject a composition comprising an adeno-associated virus (AAV) comprising an AAV capsid, and a minigene having AAV inverted terminal repeats and a Sec10, exocyst nucleic acid sequence encoding a functional Sec10 protein, operatively linked to regulatory sequences that direct expression of Sec10 in the epithelial cells,
wherein said treatment enhances repair or regeneration of the tubular epithelial cells.

2. The method according to claim 1, wherein the AAV capsid is an AAV8, AAV9, AAV6, AAV5 or AAV.rh8 capsid.

3. The method according to claim 1, wherein the Sec10, exocyst nucleic acid sequence is selected from the nucleic acid sequence of SEQ ID NO: 1 and a fragment thereof.

4. The method according to claim 1, wherein the damage to the tubular epithelial cells is caused by Acute Tubular Necrosis (ATN).

5. A method comprising over-expressing Sec10 in mammalian kidney tubular epithelial cells.

6. The method according to claim 5, wherein the overexpressing of Sec10 in kidney tubular epithelial cells occurs prior to damage thereto and prevents the onset of damage to renal epithelial cells.

7. The method according to claim 5, wherein the overexpression of Sec10 occurs in damaged epithelial cells and enhances repair thereof.

8. The method according to claim 1, wherein the tubular epithelial cells are damaged prior to delivery and wherein the method enhances repair of the damaged epithelial cells.

9. The method according to claim 1, wherein the inverted terminal repeats are from AAV2.

10. The method according to claim 9, wherein the AAV is AAV2/8, AAV2/5, AAV2/9, or AAV2/6.

11. A method for treating a mammalian subject having damage to the tubular epithelial cells, the method comprising:
delivering to the tubular epithelial cells via retrograde injection into the ureter of the subject a composition comprising an adeno-associated virus (AAV) comprising an AAV8 capsid, and a minigene having AAV2 inverted terminal repeats and a human Sec10, exocyst nucleic acid sequence which encodes a functional Sec10, operatively linked to regulatory sequences that direct expression of Sec10 in the epithelial cells, and a physiologically compatible carrier,
wherein said treatment enhances repair or regeneration of the tubular epithelial cells.

12. The method according to claim 1, wherein said subject has autosomal dominant polycystic disease.

13. The method according to claim 1, wherein said subject has had a kidney transplant.

14. (canceled)


SEQUENCE LISTING (2 sequences)

SEQ ID NO: 1 - DNA, 8522 bp, Homo sapiens
acttccggcgtatgaggcggtgacaatgggagcagcgcgggcggcgccgggaggcagctgacaagcgtttgcggcttcgcttcatggccgctctcccgcccctcctgggatctgtggggagctggggagcccgcagcggcccggagccggagctggcgagccgagcggagacctgtgcgccgcgcctctgaggcgcagcatgtgaagcggagacggcatccagtggggggcgagcctctcagccggccgggatggctaccacggccgagctcttcgaggagccttttgtggcagatgaatatattgaacgtcttgtatggagaaccccaggaggaggctctagaggtggacctgaagcttttgatcctaaaagattattagaagaatttgtaaatcatattcaggaactccagataatggatgaaaggattcagaggaaagtagagaaactagagcaacaatgtcagaaagaagccaaggaatttgccaagaaggtacaagagctgcagaaaagcaatcaggttgccttccaacatttccaagaactagatgagcacattagctatgtagcaactaaagtctgtcaccttggagaccagttagagggggtaaacacacccagacaacgggcagtggaggctcagaaattgatgaaatactttaatgagtttctagatggagaattgaaatctgatgtttttacaaattctgaaaagataaaggaagcagcagacatcattcagaagttgcacctaattgcccaagagttaccttttgatagattttcagaagttaaatccaaaattgcaagtaaataccatgatttagaatgccagctgattcaggagtttaccagtgctcaaagaagaggtgaaatctccagaatgagagaagtagcagcagttttacttcattttaagggttattcccattgtgttgatgtttatataaagcagtgccaggagggtgcttatttgagaaatgatatatttgaagacgctggaatactctgtcaaagagtgaacaaacaagttggagatatcttcagtaatccagaaacagtcctggctaaacttattcaaaatgtatttgaaatcaaactacagagttttgtgaaagagcagttagaagaatgtaggaagtccgatgcagagcaatatctcaaaaatctctatgatctgtatacaagaaccaccaatctttccagcaagctgatggagtttaatttaggtactgataaacagactttcttgtctaagcttatcaaatccattttcatttcctatttggagaactatattgaggtggagactggatatttgaaaagcagaagtgctatgatcctacagcgctattatgattcgaaaaaccatcaaaagagatccattggcacaggaggtattcaagatttgaaggaaagaattagacagcgtaccaacttaccacttgggccaagtatcgatactcatggggagacttttctatcccaagaagtggtggttaatcttttacaagaaaccaaacaagcctttgaaagatgtcataggctctctgatccttctgacttaccaaggaatgccttcagaatttttaccattcttgtggaatttttatgtattgagcatattgattatgctttggaaacaggacttgctggaattccctcttcagattctaggaatgcaaatctttattttttggacgttgtgcaacaggccaatactatttttcatctttttgacaaacagtttaatgatcaccttatgccactaataagctcttctcctaagttatctgaatgccttcagaagaaaaaagaaataattgaacaaatggagatgaaattggatactggcattgataggacattaaattgtatgattggacagatgaagcatattttggctgcagaacagaagaaaacagattttaagccagaagatgaaaacaatgttttgattcaatatactaatgcctgtgtaaaagtctgtgcttacgtaagaaaacaagtggagaagattaaaaattccatggatgggaagaatgtggatacagttttgatggaacttggagtacgttttcatcgacttatctatgagcatcttcaacaatattcctacagttgtatgggtggcatgttggccatttgtgatgtagccgaatataggaagtgtgccaaagacttcaagattccaatggtattacatctttttgatactctgcatgctctttgcaatcttctggtagttgccccagataatttaaagcaagtctgctcaggagaacaacttgctaatctggacaagaatatacttcactccttcgtacaacttcgtgctgattatagatctgcccgccttgctcgacacttcagctgagattgaatttacaaaggaattcagtgtcagttcctttacagaggaatgtcttatacttcagcagccctcggttgatagaaagcacaggagataccttatgacacagccaacattttgtgaaacaatgactggaacaaaacagcagccatacttacctttgaggttttatttaaagtttggataccactagctatattttgcttttttcccctcacattgaattttaattccattcttgaatgtagaaatttcagattctctaaaactacatgtcactgtttttatcctagaaaatgttgctgtcagaaggcaaaggaaatgttaccagtgttttcggttcttgtacttttaacatattccatttagaaattttgccattctgttttccattaataataggtgaaatacaggaaaactacatttgttattcctcagtttttaatgaccttttcagcatcaattgttaatcagattattttaggttttcgtaaataatttttttgcctctttcaaaaggttaacaattaagcatactttctgcagttggttgattggatttttttctgaggtacagcattaatactagtccaaaaaatgtcataaactgaactaaaatgatgaactattttatgtagacattaggagtggatcggaatacttctgctttctgggtaaaacttaaaagtttactatttcttatttggtaaatagattttaagccaattctagtaagaaattaataaaactaccttattttgtatttcacttaaggtggaggaccttaactaaaggaccatatttattcattattttaatattataagggaagtaaaaaaaagtgaggtatagtctaaatggtgcatataggaaatactgacagtgtttagcaacatgcagccctttgagatttctgtcgtaatgctaaacttgaataagatggaatggctgaacatgtggttagtcttttattttaagaagaattgagaattgatagatttggagatgagctttgcaaaggctgtttgcttttcatgtctataggtctgtcattgtcctttttcaaagcatttctgaagttattcctacttggatatagttaatggaattggcttaatttgatgacataataaatcacttataaaattttaaatatcaagtgaaaatttagaaaggccattactattctataaaccttataaacttgctctgggagaatgcattctaaattatatatagtgtttcagctcccattgtggtgttcatagtcttctaggaacagataaacttaagtattcaattcactcttggcattttttctttaatataggctttttagcctatttttggaaaactgcttttcttctgagaaccttattctgaatgtcatcaactttaccaaaccttctaagtccagagctaacttagtactgtttaagttactattgactgaattttcttcattttctgtttagtccagtgttaccaaggtaagctggggaatgaagtataccaacttctttcagagcattttaggacattatggcagctttagaaggctgtcttgtttctagccaagggagagccagcgcaggttttggatactagagaaagtcatttgcttgtactattgccattttagaaagctctgatgtgaattcaaattttacctctgttacttaaagccaacaattttaaggcagtagttttactggccatttaagctctttgtacagtgtcaatttgtaaaaaagaaaaaacgaaaaaaaatctcaaataaaacatgagataacattttaagacttccaaatcagagaagtgcttcaaattattttgtttggattaatttttaaaatgtaaagcatatacttgttgcttagttattttgtttattattttcatgtttgagttctgtgcaatattttctattatgtccgttgataggacagtgacaaaattcgtaagtgaatacttaatttaaaagtgttaacattaatgctcaataaacaaattccctgcagttgttttttttatttcactctttttatattttagagccatccatcctgaattttataaatgattttttatttaaaaatttgagatttttaaaatttttcacagcacaggtcttctcattttcttttttagcaaaaaatacttgactatataaacaagactagaaatttatcctaaacagatggtctttctctgagggaaaaaaaaaaatgtgtcatgtgaggcaatatgcattctttgtattctgattttttaagtgttttcatatttatagccatcacaaaattttgcccaaagtgtatggctcagaagagatgatcaataactaaaatgtttttcgttttgcacatggtaaaattttaggtagagagtggagctttgtataattggtaactttaaccaacatttacgtaagtacggtctttgcagtattacacaataccgcatgagctaatatgcagtatttaggtaatgcattgaatgaagttgaaaatatgctcttaacacactgtgcattgtttttctaaaattgagaaatatctgaaatacagaatacgaatttaataaaggaagcctaccttatgtaaattatgatgttgaaaatgacagtcaaccagagtagatgaagtgtccttatgaagaattattaaatagtagctggatggatctttagatggatgaccacctgaccattccaggacaatttttggtcccattttgaggattgccatagttgcaagtccttaggatcttccttgttttaaaaaactatcaaagtaggttaaattaaaatctaatttaaattaggttaatttatattacattgtagatcattcttttttcccccctctttcacttctaaatattttgctttctaaatactagccttctggttcacagtgaaataatttaattatcaatcagtagattgattccaagtggagaattcactgcttccctaaaatgcgtttttcttacctgtaatggtgaatatttacaaggatctggataaagagattttgatttttgaaatgccacaaaaccccttaaagtggtcaagtacctaaggtagtggctaaggcatgcttgaactgtgtttgagagacagcagttcagtcagtacataatttgttggtgactaacaaagctcacatctgggagattaactttttttaacgcaaaaatattctgcacattacccatatattgttgaattttacatacaaagcattttgtgctttcattcaattagtttaactatattcagtttctgtattatttctctctgatctatcctgtaattattctatactttagaagaaaagatcagttcatattgttagactgaataattctacatgaagttaaaagttagcaatttaaaaaattttgctgcataataaagataattatagttacatgtattgatttaagtctcacacactgtgctaataagtcctttgcacatattattggattctagagcaaccctaggatttgggtactattatttccatttgacagatgaaaaacctaatgcatagaaagataagtagaccagtcaaggtctacagctagtaaaatgctcagattgaaacctaaatcttttctcactcacttttaggttctatatgctattctccttacgtaacgttaaatagatcctttagtttgataggttcattagttttgcttaatcgttgcttccaatatgattcttattgtagtatttatattacaaagttggcatacagggtcaatatctaatttactgttgtgtttttcttaatatgttcaatgtatggtcatactttttaaatttgattttttaaatattataagtggtcatgaaattttaatgttaagtttgtacctaatagaaatatgtctatcacaaatctttattggtattattttatatacttttccagcttgataattttcacaatagaaagagaccaaatattttgtactatattttgtgacatagaagtaaatgacaactctaccttggaagatatgaagaaatcagtgtttttatgttcaaaaactattttaaatgtcataggcttgccaattgtgttaactatttagtacattggtagtttttaaatgactaatgctggagtgaaaatgtaaggcaaaatatttagtatattaacagtagatagtcattttcatgtaatcagaattgcatgttaagggttttaaaagctgtcataactcttaccttttatttctaccagtgatcaatcatttctctttacaaatttagggaggaaacaactgttcaaatgtataggggacagaaacctgacatttagagaacagaaattttaattgccaaattaaagcagctaggctgcccctgggcttaacaaaattcctatttttgaactagacccgaggtattttatcattattatttgataggtaccattaacctttgttttgtgattatcgacttttggggggaaagatgagtaattaaacacactcgcccattttctaagtggtttagaaggttgcagtctgcctcaccatacaaagccagtttaaataggaatatatcacatcatacttcacatagcagtcaacctcagaggaaagtcatttattcagaaggcatagattttgctagacagcagtgatgcattacaaatatataattatttgcactaatatttgagcaggtggaatggattagaacatggtcaacatcatgctgctgataacatgtatttttctatattaatatgtactgtgcaacatgtattaaaattgtcaataattcattttagccaagatactaatatttatattactgtgtttaagagacagtatccaactgagccacttttaatttaaaagtctgaaactggaattaagtttactttcaaaaatcacttaaattactttataatcagtgaaaggtcagtaaaatgtagaattataagtgcctagtgacttaataggaggaaatatggagggttaaggaaggaagtaggaacctggaataagaaagctaagtttcatttatagttccaatgagtgactttctttgttttcaccactgggaatcttaaatgaagaaagtgtcgtatcccttccagttatctttatgttaataaactaagtgaagtttggatagaactaacaaaggaattagtctccattggtggacttccagagttttgagattaaactttgagaggtgagaagccttcctgatgcccctgtaatcttaaattggcaggtgcctctaagtgaggccatccctctttgcctgcctatcattttaatctttatactgtaactggttcctttcaactctggccttttattgtagcacttttatcaagtctgcagcttcccagctacttgggaggctgaggtgggaagaacacttgaagctaggagttcaagaccactctgggcaacatagtgagtccctgtttttagaaaaataaataacctttaggtttttgctatagaaaatatccaaatagagtctggacaatgctggatgtaaagaaaactgtgaagctacctctaagcatattgtttgtgaatactgtgtaccatttagaggataacatgggggattcacatgaagttatgtgaaaatattttataaggaataatacctaagtgcctctcttaagttctccagtagaatgtattttcaatttttgccacaagggggagaagttatttttatagtaatatgtacatttttttattttaaaaatagaattcacacaactttacagcatctatgaacattttgctatcccaattatgtgtgtgtgtgcatgtgtgtaaacaagattctgaaagtcttgcatgagaaaaatttcaaaggaaattaataacaaaaccaaaacagttttgaagaacatgtgcacagtggacacaggaattgaacaaagtttaccatgattaacattatgttttcttagttatatatcacattgaattcctttatcaactctaataagaatctactgattagtgttctacattctgctttttaaagaaaaatttttgaaacaatcagacttaacagaagggttgtaaatactgtacaaataaaatttttcccttttgagaataaaaaaaaaaaaa

SEQ ID NO: 2 - protein, 708 aa, Homo sapiens
MATTAELFEEPFVADEYIERLVWRTPGGGSRGGPEAFDPKRLLEEFVNHIQELQIMDERIQRKVEKLEQQCQKEAKEFAKKVQELQKSNQVAFQHFQELDEHISYVATKVCHLGDQLEGVNTPRQRAVEAQKLMKYFNEFLDGELKSDVFTNSEKIKEAADIIQKLHLIAQELPFDRFSEVKSKIASKYHDLECQLIQEFTSAQRRGEISRMREVAAVLLHFKGYSHCVDVYIKQCQEGAYLRNDIFEDAGILCQRVNKQVGDIFSNPETVLAKLIQNVFEIKLQSFVKEQLEECRKSDAEQYLKNLYDLYTRTTNLSSKLMEFNLGTDKQTFLSKLIKSIFISYLENYIEVETGYLKSRSAMILQRYYDSKNHQKRSIGTGGIQDLKERIRQRTNLPLGPSIDTHGETFLSQEVVVNLLQETKQAFERCHRLSDPSDLPRNAFRIFTILVEFLCIEHIDYALETGLAGIPSSDSRNANLYFLDVVQQANTIFHLFDKQFNDHLMPLISSSPKLSECLQKKKEIIEQMEMKLDTGIDRTLNCMIGQMKHILAAEQKKTDFKPEDENNVLIQYTNACVKVCAYVRKQVEKIKNSMDGKNVDTVLMELGVRFHRLIYEHLQQYSYSCMGGMLAICDVAEYRKCAKDFKIPMVLHLFDTLHALCNLLVVAPDNLKQVCSGEQLANLDKNILHSFVQLRADYRSARLARHFS